SEQUENCE LISTING (17 sequences)

SEQ ID NO: 1 - DNA, 18 bp, Artificial Sequence
  primer
cgtacggaattcgctagc

SEQ ID NO: 2 - DNA, 18 bp, Artificial Sequence
  primer
ggatccgagctccacgtg

SEQ ID NO: 3 - DNA, 56 bp, Artificial Sequence
  DNA aptamer T1
cgtacggaattcgctagcagcgtccacgagtgtggtgtggatccgagctccacgat

SEQ ID NO: 4 - DNA, 76 bp, Artificial Sequence
  DNA aptamer T3
cgtacggaattcgctagcacgttgacgctggtgcccggttgtggtgcgagtgttgtgtggatccgagctccacgtg

SEQ ID NO: 5 - DNA, 76 bp, Artificial Sequence; identical to SEQ ID NO: 4
  DNA aptamer T4

SEQ ID NO: 6 - DNA, 56 bp, Artificial Sequence
  DNA aptamer T10
cgtacggaattcgctagcgagtcatgtaccgtccctgtggatccgagctccacgtg

SEQ ID NO: 7 - DNA, 75 bp, Artificial Sequence
  DNA aptamer Tn1
cgtacggaattcgctagcacgttgacgctggtgcccggttgtgggcgagtgttgtgtggatccgagctccacgtg

SEQ ID NO: 8 - DNA, 56 bp, Artificial Sequence
  DNA aptamer Tn3
cgtacggaattcgctagcgtgtcaataatggtcctctgggatccgagctccacgtg

SEQ ID NO: 9 - DNA, 76 bp, Artificial Sequence
  DNA aptamer Tc2
cacgtggagctcggatccacgcgcagtgggaccaacccaagccgtggcctgccggggggctagcgaattccgtacg

SEQ ID NO: 10 - DNA, 76 bp, Artificial Sequence
  DNA aptamer Tc3
cacgtggagctcggatccacacaacactcgcaccacaaccgggcaccagcgtcaacgtgctagcgaattccgtacg

SEQ ID NO: 11 - DNA, 75 bp, Artificial Sequence
  DNA aptamer i11
cgtacggaattcgctagcacgttgacgctggtgcccggtttggtgcgagtgttgtgtggatccgagctccacgtg

SEQ ID NO: 12 - DNA, 77 bp, Artificial Sequence
  DNA aptamer i13
cgtacggaattcgctagcacgttgacgctggtgcccggttgtgggtgcgagtgttgtgtggatccgagctccacgtg

SEQ ID NO: 13 - DNA, 76 bp, Artificial Sequence
  DNA aptamer i18
cgtacggaattcgctagcacgttgacgctggtgcccggttgtggtgcgggtgttgtgtggatccgagctccacgtg

SEQ ID NO: 14 - DNA, 76 bp, Artificial Sequence
  DNA aptamer MBA
cgtacggaattcgctagccccccggcaggccacggcttgggttggtcccactgcgcgtggatccgagctccacgtg

SEQ ID NO: 15 - DNA, 76 bp, Artificial Sequence
  DNA aptamer T2
cgtacggaattcgctagccccccggcaggccacggcttgggttggtccctctgcgcgtggatccgagctccacgtg

SEQ ID NO: 16 - DNA, 18 bp, Artificial Sequence
  forward primer
cgtacggaattcgctagc

SEQ ID NO: 17 - DNA, 18 bp, Artificial Sequence
  reverse primer
cacgtggagctcggatcc

(19) United States
(12) Patent Application Publication
(10) Pub. No.: US 2016/0251660 A1
(43) Pub. Date: Sep. 1, 2016

(54) NUCLEIC ACID APTAMER CAPABLE OF SPECIFICALLY BINDING TO TEBUCONAZOLE, MEFENACET AND INABENFIDE, AND USE THEROF

(71) Applicant: KOREA UNIVERSITY RESEARCH AND BUSINESS FOUNDATION, Seoul (KR)

(72) Inventors: Man Bock Gu, Seoul (KR); Van Thuan Nguyen, Seoul (KR); Young Seop Kwon, Seoul (KR)

(73) Assignee: KOREA UNIVERSITY RESEARCH AND BUSINESS FOUNDATION, Seoul (KR)

(21) Appl. No.: 15/028,931

(22) PCT Filed: Oct. 16, 2014
(86) PCT No.: PCT/KR2014/009696

§ 371 Date: Apr. 12, 2016

(30) Foreign Application Priority Data
Oct. 16, 2013 (KR) 10-2013-0123471

(51) Int. Cl. C12N 15/115; G01N 33/53

(52) U.S. Cl. CPC C12N 15/115; G01N 33/5308; C12N 2310/16

(57) ABSTRACT

The present invention relates to a nucleic acid aptamer capable of binding to tebuconazole, mefenacet or inabenfide, and the use thereof, and more particularly, to a nucleic acid aptamer capable of binding specifically to a very small amount of tebuconazole, mefenacet or inabenfide, and a method of detecting and separating tebuconazole, mefenacet or inabenfide using the nucleic acid aptamer. The nucleic acid aptamer of the present invention, which can bind specifically to tebuconazole, mefenacet or inabenfide, enables the detection and separation of tebuconazole, mefenacet or inabenfide present in samples such as food in very small amounts, and thus is effective for the detection and removal of tebuconazole, mefenacet or inabenfide which was difficult to detect in the prior art. In addition, because the nucleic acid aptamer of the present invention can be produced at low costs, a method and kit for detecting tebuconazole, mefenacet or inabenfide, and a kit for separating tebuconazole, mefenacet or inabenfide, which use the nucleic acid aptamer of the present invention, are cost-effective and eliminate the need to develop aptamers for various targets. Thus, the nucleic acid aptamer of the present invention is highly useful.

### TECHNICAL FIELD

The present invention relates to a nucleic acid aptamer capable of binding to tebuconazole, mefenacet and inabenfide, and the use thereof, and more particularly, to a nucleic acid aptamer capable of binding specifically to tebuconazole, mefenacet and inabenfide, and a method of detecting and removing tebuconazole, mefenacet and inabenfide using the nucleic acid aptamer.

### BACKGROUND ART

Tebuconazole is a triazole derivative that is widely used to prevent red pepper anthracnose. The US FDA considers this fungicide safe for human use, but the USA Environmental Protection Agency (EPA) classifies tebuconazole as a group C carcinogen (possible human carcinogen), and the Swedish Chemicals Agency classifies tebuconazole as a potential endocrine disrupting chemical. Thus, tebuconazole is still considered risky. Recently, tebuconazole was detected in red pepper powder (produced by a big food company) in an amount exceeding an allowable limit, and thus prohibition of the distribution and sale of the red pepper powder was imposed.

Mefenacet is a kind of herbicide and was not reported to carcinogenic. However, it is known that mefenacet can cause allergic reactions if the skin is exposed to mefenacet for a long period of time, and mefenacet can cause inflammation if it enters eyes. In addition, if mefenacet remains in the environment for a long period of time, it will have fatal effects on bees to cause ecological disturbance.

Inabenfide is a plant growth inhibitor, and long-term intake of inabenfide has been prohibited. In addition, it is recommended to avoid the contact of inabenfide with the skin or eyes or the inhalation of inabenfide. However, inabenfide is used in a very wide range of applications, and for this reason, can cause environmental pollution problems. If agricultural and marine products in which substances such as tebuconazole, mefenacet or inabenfide remain are taken into the human or animal body for a long period of time, these products can cause internal organ dysfunction, DNA damage, damage to the nervous system and development, cancer, delivery of deformed babies, miscarriage, or the like. Thus, in order to prevent the misuse of tebuconazole, mefenacet or inabenfide and ensure the safety of foods, it is needed to develop a method for detecting pesticides remaining in soil, water, or agricultural and marine products.

In the prior art, for the detection of residual pesticides, a method was mainly used which comprises preparing different test solutions according to components in which residual pesticides are dissolved, and then extracting and purifying residual pesticides from the test solutions, followed by quantification by liquid chromatography. However, in the detection method according to the prior art, the number of test methods that are performed according to dissolved substances is small, and it is complicated to each of test solutions. In addition, it is difficult to achieve accurate quantification, because tebuconazole, mefenacet or inabenfide can be lost during the extraction and purification processes. In particular, it is important to analyze tebuconazole, mefenacet and inabenfide which are present in water supply sources, rivers, and water resources that are used as mineral water or the like. However, there are disadvantages in that, because the detection method according to the present invention relies mainly on instrumental analysis, it is almost impossible to perform the analysis in situ, and the analysis is costly.

Meanwhile, aptamers refer to single-stranded DNA or RNA molecule structures that are generated from random nucleic acid libraries having a diversity of about 10¹²⁻¹⁴and that have a high specificity and affinity for particular targets. In addition, unlike antibodies that are used as probes in the sensor field, aptamers have excellent thermal stability because they are nucleic acid structures. Also, because these aptamers are synthesized in vitro and do not require animals or cells for their synthesis, these are economical in terms of the production cost. In addition, because materials to be targeted by these aptamers are not limited, aptamers for various targets, including small-molecule organic chemical substances such as environmental hormones, antibiotics and residual drugs, bacteria, viruses or the like, can be synthesized from biomolecular substances such as proteins or amino acids. Thus, aptamers for various target materials have been synthesized, and due to the properties of aptamers that binds to target materials with specificity and strong affinity, many studies on the use of aptamers in new drug development, drug delivery systems and biosensors have recently been conducted. Thus, aptamers are very suitable for use in methods for detecting very small amounts of residual pesticides, and can also be applied to detect particular residual pesticides through nano-biotechnology.

The most important factor in the development of aptamers is to distinguish target-bound DNA (or RNA) from unbound DNA. For this distinction, studies have generally been conducted to distinguish between DNAs by immobilizing a target or a DNA random library. However, the biggest difficulties in this immobilization method are that the immobilization yield can be low and that the analysis of the immobilization yield is cost- and time-consuming. In addition, in the immobilization method, the possibility for a separation material (magnetic bead, column, etc.) being bound directly to DNA used for immobilization cannot be entirely excluded, and the possibility of loss of a DNA pool which may occur when the DNA bound to a target immobilized to the separation material is separated again remains as a limit and problem of the immobilization method. In particular, a low DNA immobilization rate problem which may occur when a DNA library is immobilized is related directly to the loss of a DNA pool which is the biggest loss to be avoided during an aptamer development process and thus serves as an upper limit. Moreover, it is difficult to develop aptamers by the immobilization method for heavy metal ions which cannot be immobilized, and thus the immobilization method may be limited in target selection. However, the above-described limits can be overcome by the use of immobilization-free aptamer development technology. Further, because a target-binding site is not limited, it is possible to reduce the number of repetitions of a selection process required for development of an aptamer. For this reason, in order to invent a technique by which an aptamer can be developed through a immobilization-free method, a microelectromechanical system (MEMS), capillary electrophoresis, etc. have been conventionally used, but expensive equipment, complexity in use of devices, necessity of skilled manpower, and the like still remain as problems. Meanwhile, graphene is a two-dimensional carbon structure having excellent thermal stability, electrical characteristics, and strength and is bound to the base moiety of a single-stranded DNA by π-stacking, and thus a wide range of studies using such characteristics have been conducted. However, when aptamers is to be developed by the immobilization-free method using graphene-based SELEX, there is a shortcoming in that the development of aptamers for various targets is time-consuming. This is because it is required to determine a counter target for each major target and to perform the analysis of the major target and the counter target, and SELEX for each target.

Accordingly, the present inventors have made extensive efforts to overcome the above-described problems occurring in the prior art, and as a result, have developed a nucleic acid aptamer (nucleic acid structure), which shows a high specificity and affinity for tebuconazole, mefenacet and inabenfide, by use of immobilization-free graphene SELEX. In addition, the present inventors have prepared a composition for a gold nanparticle-based colorimetric assay, which contains the nucleic acid aptamer that binds specifically to tebuconazole, mefenacet or inabenfide, and have found that the use of the composition can effectively detect or remove a very small amount of tebuconazole, mefenacet or inabenfide, which remains in food or the like, thereby completing the present invention.

The above information disclosed in the Background Art section is only for enhancement of understanding of the background of the present invention, and therefore it may contain information that does not form the prior art that is already known to a person of ordinary skill in the art.

### DISCLOSURE OF INVENTION

### Technical Problem

It is an object of the present invention to provide a nucleic acid aptamer capable of detecting even very small amounts of tebuconazole, mefenacet and inabenfide, and the use thereof.

### Technical Solution

To achieve the above objects, the present invention provides a nucleic acid aptamer capable of binding specifically to tebuconazole, mefenacet or inabenfide, which has a nucleotide sequence selected from the group consisting of SEQ ID NOs: 3 to 15.

The present invention also provides a method for detecting tebuconazole, mefenacet or inabenfide, the method comprising a step of bringing the nucleic acid aptamer into contact with a sample.

The present invention also provides a composition for detecting tebuconazole, mefenacet or inabenfide, which contains the above-described aptamer.

The present invention also provides a sensor for detecting tebuconazole, mefenacet or inabenfide, which contains the above-described aptamer.

The present invention also provides a kit for detecting tebuconazole, mefenacet or inabenfide, which contains the above-described aptamer.

The present invention also provides a method of separating tebuconazole, mefenacet or inabenfide from a sample using the above-described aptamer.

The present invention also provides a composition for separating tebuconazole, mefenacet or inabenfide, which contains the above-described aptamer.

The present invention also provides a kit for detecting tebuconazole, mefenacet or inabenfide, which contains the above-described aptamer.

Other features and embodiments of the present invention will be more apparent from the following detailed descriptions and the appended claims.

### BRIEF DESCRIPTION OF THE DRAWINGS

FIG. 1 is a schematic view showing a process of producing a target-specific aptamer using an S-SELEX MT method developed based on immobilization-free graphene SELEX.

FIG. 2 is a graph showing an increase in the amount of an ssDNA binding to tebuconazole, mefenacet and inabenfide, obtained from each selection round.

FIG. 3 is a schematic view illustrating the principle by which target materials are detected by the binding between an aptamer and gold nanoparticles.

FIGS. 4 to 16 show aptamer secondary structures of the present invention, which are represented by SEQ ID NOs: 3 to 15 and can bind specifically to tebuconazole (T), mefenacet (MBA) or inabenfide (I).

FIG. 17 shows the results of observing gold nanoparticle-based color changes in various samples using nucleic acid aptamer T1.

FIG. 18 shows the results of observing gold nanoparticle-based color changes in various samples using aptamer MBA.

FIG. 19 shows the results of observing gold nanoparticle-based color changes in various samples using aptamer i13.

FIG. 20 shows the results of observing gold nanoparticle-based color changes in various samples using aptamer T2.

FIG. 21 shows the results of observing gold nanoparticle-based color changes for various concentrations of tebuconazole using aptamer T1.

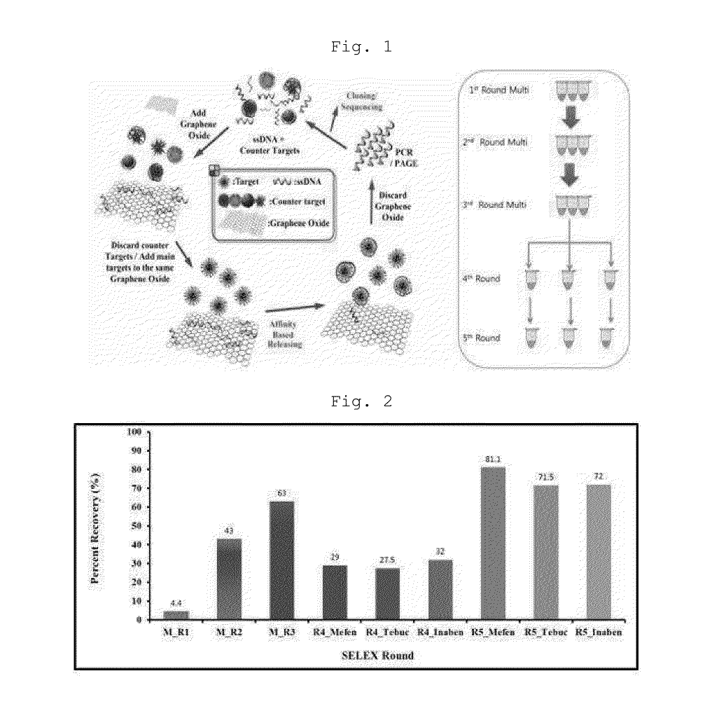

### BEST MODE FOR CARRYING OUT THE INVENTION

Unless defined otherwise, all technical and scientific terms used herein have the same meaning as commonly understood by one of ordinary skill in the art to which the invention pertains. Generally, the nomenclature used herein and the experiment methods, which will be described below, are those well known and commonly employed in the art.

The definition of main terms used in the detailed description of the invention is as follows.

As used herein, the term “nucleic acid aptamer” refers to a small single-stranded oligonucleotide that can specifically recognize its target material with high affinity.

As used herein, the term “sample” refers to a composition that contains or is assumed to contain tebuconazole, mefenacet or inabenfide and will be analyzed. The sample may be a sample collected from any one or more of, but not limited to, liquid, soil, air, food, waste, animal intestines, and animal tissues. Herein, examples of the liquid may be serum, blood, urine, water, tears, sweat, saliva, lymph, and cerebrospinal fluid. Examples of the water include river water, seawater, lake water, and rain water. Examples of the waste include sewage, waste water, and the like. The animals include the human body. Further, examples of the animal and plant tissues include mucous membranes, skin, cortices, hair, scales, eyes, tongues, cheeks, hooves, beaks, snouts, feet, hands, mouths, nipples, ears, noses, etc.

As used herein, the term “GO SELEX process” refers to a method of identifying a DNA sequence specific for each molecule by selecting and amplifying a DNA or RNA having a high affinity for a particular molecular from a group of randomly synthesized DNAs or RNAs (J. W. Park, R. Tatavarty, D. W. Kim, H. T. Jung and M. B. Gu (2012), Immobilization-free screening of aptamers assisted by graphene oxide, Chemical Communications, 48, 15, 2071-2073).

In one aspect, the present invention is directed to a nucleic acid aptamer capable of binding specifically to tebuconazole, mefenacet or inabenfide, which has a nucleotide sequence selected from the group consisting of SEQ ID NOs: 3 to 15.

Aptamer T1:
(SEQ ID NO: 3)
5′-CGTACGGAATTCGCTAGCAGCGTCCACGAGTGTGGTGTGGATCCGA
GCTCCACGAT-3′
Aptamer T3:
(SEQ ID NO: 4)
5′-CGTACGGAATTCGCTAGCACGTTGACGCTGGTGCCCGGTTGTGGTG
CGAGTGTTGTGTGGATCCGAGCTCCACGTG-3′
Aptamer T4:
(SEQ ID NO: 5)
5′-CGTACGGAATTCGCTAGCACGTTGACGCTGGTGCCCGGTTGTGGTG
GAGTGTTGTGTGGATCCGAGCTCCACGTG-3′
Aptamer T10:
(SEQ ID NO: 6)
5′-CGTACGGAATTCGCTAGCGAGTCATGTACCGTCCCTGTGGATCCGA
GCTCCACGTG-3′
Aptamer Tn1:
(SEQ ID NO: 7)
5′-CGTACGGAATTCGCTAGCACGTTGACGCTGGTGCCCGGTTGTGGGC
GAGTGTTGTGTGGATCCGAGCTCCACGTG-3′
Aptamer Tn3:
(SEQ ID NO: 8)
5′-CGTACGGAATTCGCTAGCGTGTCAATAATGGTCCTCTGGGATCCGA
GCTCCACGTG-3′
Aptamer Tc2:
(SEQ ID NO: 9)
5′-CACGTGGAGCTCGGATCCACGCGCAGTGGGACCAACCCAAGCCGTG
GCCTGCCGGGGGGCTAGCGAATTCCGTACG-3′
Aptamer Tc3:
(SEQ ID NO: 10)
5′-CACGTGGAGCTCGGATCCACACAACACTCGCACCACAACCGGGCAC
CAGCGTCAACGTGCTAGCGAATTCCGTACG-3′
Aptamer i11:
(SEQ ID NO: 11)
5′-CGTACGGAATTCGCTAGCACGTTGACGCTGGTGCCCGGTTTGGTGC
GAGTGTTGTGTGGATCCGAGCTCCACGTG-3′
Aptamer i13:
(SEQ ID NO: 12)
5′-CGTACGGAATTCGCTAGCACGTTGACGCTGGTGCCCGGTTGTGGGT
GCGAGTGTTGTGTGGATCCGAGCTCCACGTG-3′
Aptamer i18:
(SEQ ID NO: 13)
5′-CGTACGGAATTCGCTAGCACGTTGACGCTGGTGCCCGGTTGTGGTG
CGGGTGTTGTGTGGATCCGAGCTCCACGTG-3′
Aptamer MBA:
(SEQ ID NO: 14)
5′-CGTACGGAATTCGCTAGCCCCCCGGCAGGCCACGGCTTGGGTTGGT
CCCACTGCGCGTGGATCCGAGCTCCACGTG-3′
Aptamer T2:
(SEQ ID NO: 15)
5′-CGTACGGAATTCGCTAGCCCCCCGGCAGGCCACGGCTTGGGTTGGT
CCCTCTGCGCGTGGATCCGAGCTCCACGTG-3′

The nucleic acid aptamer is provided in the form of a single-stranded DNA or RNA. In the present invention, if the nucleic acid is RNA, “T” in the nucleic acid sequence is to be read as “U”. It will obvious to a person of ordinary skill in the art that these sequences fall within the scope of the present invention.

The nucleic acid aptamer of the present invention may be a nucleic acid aptamer having any nucleotide sequence, which is selected by the GO SELEX (Systematic Evolution of Ligands by Exponential Enrichment) process and is capable of binding specifically to tebuconazole, mefenacet or inabenfide.

More specifically, the nucleic acid aptamer capable of binding specifically to tebuconazole, mefenacet or inabenfide of the present invention may be produced by a method comprising the steps of:

a) mixing a single-stranded nucleic acid pool, which includes PCR primer regions at both ends and has 30-50 random nucleotides in its center, with a target material or a counter-target material in a buffer solution, and inducing the binding therebetween at room temperature;

b) reacting the mixture with graphene to remove a single-stranded nucleic acid bound to the target material or unbound to the counter-target material;

c) amplifying the single-stranded nucleic acid, which results from step b) and binds specifically to the target material, by PCR using the PCR primer regions;

d) repeatedly performing a graphene-based selection process and a counter-selection process on the single-stranded nucleic acid, which binds specifically to the target material, by use of the target material and the counter-target material; and

e) removing a target-nonspecific single-stranded nucleic acid which binds to the counter-target material in the graphene-based counter-selection process, and inducing a target-induced conformational change in the target-specific single-stranded nucleic acid bound to the graphene, thereby separating a target-specific aptamer from the graphene.

Step d) of the method for producing the DNA aptamer may further comprise a step of performing PCR using a fluorescein-labeled primer of the primer pair, and then separating the modified single-stranded DNA by electrophoresis.

In an example of the present invention, in order to produce an aptamer that binds specifically to tebuconazole, mefenacet or inabenfide, a nucleic acid aptamer that binds to tebuconazole, mefenacet or inabenfide was screened by the GO SELEX (Systematic Evolution of Ligands by Exponential enrichment) process, and then analyzed by a gold nanoparticle-based colorimetric assay. As a result, it was found that aptamers having nucleotide sequences represented by SEQ ID NOs: 3 to 15 bind specifically to each of tebuconazole, mefenacet and inabenfide.

In another aspect, the present invention is directed to a method for detecting tebuconazole, mefenacet or inabenfide, the method comprising a step of bringing the nucleic acid aptamer into contact with a sample.

The sample may be a sample collected from any one or more of water, soil, waste, food, animal intestines, and animal and plant tissues, but is not limited thereto. Herein, examples of the water include river water, seawater, lake water, and rain water, examples of the waste include sewage, waste water, and the like, and the animals include the human body.

In an example of the present invention, it was found by a gold nanoparticle-based colorimetric assay that the nucleic acid aptamer according to the present invention binds specifically to tebuconazole, mefenacet or inabenfide.

The gold nanoparticle-based colorimetric assay has recently been considered as a new alternative for on-site detection in that it is conveniently prepared, is simply operated, and enables color changes to be visually observed [Zhao, W., Brook, M.A., Li, Y., 2008a. ChemBioChem 9, 2363-2371]. Gold nanoparticle-based aptamer sensors include two types. One type comprises an aptamer immobilized on the modified surface of gold nanoparticles by covalent bonding or the like, and uses the property that aggregation of the gold nanoparticles occurs due to a reduction in the distance between two or more of the gold nanoparticles in the presence of a target material to thereby change the color of the gold nanoparticle solution from a red color to a blue-based color. The other one type comprises an aptamer physically adsorbed onto the unmodified surface of gold nanoparticles, and uses the property that the adsorbed aptamer is detached from the surface of the gold nanoparticles in the presence of a target material due to its binding to the target to thereby change the color of the gold nanoparticles. A solution of pure gold nanoparticles has a red color, and if a target is added to gold nanoparticles after an aptamer was physically adsorbed onto the gold nanoparticles, the aptamer will bind to the target because the affinity between the aptamer and the target is greater than the affinity between the aptamer and the gold nanoparticles [Y. S. Kim, et al., A novel colorimetric aptasensor using gold nanoparticle for a highly sensitive and specific detection of oxytetracycline, Biosensors and Bioelectronics, Volume 26, Issue 4, 15 2010]. At this time, when NaCl is added, a color change caused by aggregation of the gold nanoparticles can be observed in a tube containing the target added thereto. On the contrary, in a tube free of the target, no color change can be observed because the aptamer interferes with the NaCl-induced aggregation of the gold nanoparticles. Based on such properties, the use of aptamer-gold nanoparticles can detect a target material. In addition, when the absorbance of gold nanoparticle solutions is measured by a spectrophotometer, a solution of pure gold nanoparticles shows the highest absorbance at 520 nm, whereas it shows the highest absorbance at 650 nm after the change changed to a blue color. Thus, the value obtained by dividing the absorbance value at 650 nm by the absorbance value at 520 nm increases in proportion to the degree of aggregation induced by the target. In an example of the present invention, whether the aptamer screened in the present invention binds specifically to tebuconazole, mefenacet or inabenfide was analyzed by the gold nanoparticle-based colorimetric assay as described above.

Specifically, the present invention comprises an aptamer immobilized on the modified surface of gold nanoparticles by covalent bonding or the like, and uses the property that aggregation of the gold nanoparticles occurs due to a reduction in the distance between two or more of the gold nanoparticles in the presence of a target material to thereby change the color of the gold nanoparticle solution from a red color to a blue-based color. Alternatively, the present invention comprises an aptamer physically adsorbed onto the unmodified surface of gold nanoparticles, and uses the property that the adsorbed aptamer is detached from the surface of the gold nanoparticles in the presence of a target material due to its binding to the target to thereby change the color of the gold nanoparticles. Using such properties, tebuconazole, mefenacet or inabenfide can be detected.

In an example of the present invention, a gold nanoparticle-based colorimetric assay was performed on aptamers T1, i13 and MBA having nucleic acid sequences represented by SEQ ID NOs: 3, 9 and 14, which show the highest affinity among the nucleic acid aptamers represented by SEQ ID NOs: 3 to 4. As a result, it was shown that the aptamers did bind specifically to tebuconazole, mefenacet and inabenfide, respectively.

Therefore, in still another aspect, the present invention is directed to a composition for detecting tebuconazole, mefenacet or inabenfide, which contains the above-described aptamer that binds specifically to tebuconazole, mefenacet or inabenfide.

The aptamer of the present invention may be chemically synthesized by any method already known in the art.

The aptamer of the present invention may be one wherein a sugar residue (e.g., ribose or deoxyribose) of each nucleotide has been modified to increase the affinity of the aptamer for tebuconazole, mefenacet or inabenfide, the stability of the aptamer, and the like. As examples of the site to be modified in a sugar residue, one having the oxygen atom at the 2′-position, 3′-position and/or 4′-position of the sugar residue replaced with another atom, and the like can be mentioned. As examples of the modification, fluoration, O-alkylation (e.g., O-methylation, O-ethylation), O-arylation, S-alkylation (e.g., S-methylation, S-ethylation), S-arylation, and amination (e.g., —NH₂)canbementioned. Such alterations in the sugar residue can be performed by a method known per se (For example, see Sproatet al., (1991) Nucle. Acid. Res. 19, 733-738; Cotton et al., (1991) Nucl. Acid. Res. 19, 2629-2635; Hobbs et al., (1973) Biochemistry 12, 5138-5145).

The aptamer of the present invention may also have a nucleic acid base (e.g., purine or pyrimidine) altered (e.g., chemical substitution) to increase its affinity for tebuconazole, mefenacet or inabenfide, or the like. Examples of such alterations include pyrimidine alteration at 5-position, purine alteration at 6- and/or 8-position(s), alteration with an extracyclic amine, substitution with 4-thiouridine, and substitution with 5-bromo or 5-iodo-uracil.

Also, the phosphate group contained in the aptamer of the present invention may be altered to confer resistance to nuclease and hydrolysis. For example, the P(O)O group may be replaced with P(O)S (thioate), P(S)S (dithioate), P(O)NR₂(amidate), P(O)R, R(O)OR′, CO or CH₂(formacetal) or 3′-amine(-NH—CH₂—CH₂—), wherein each unit of R or R′ is independently H or a substituted or unsubstituted alkyl (e.g., methyl,ethyl). The linking group is, for example, —O—, —N— or —S—, and nucleotides can bind to an adjoining nucleotide via these linking groups.

The alterations may also include alterations such as capping at 3′ and 5′. An alteration can further be performed by adding to an end a polyethyleneglycol, amino acid, peptide, inverted dT, nucleic acid, nucleosides, myristoyl, lithocolic-oleyl, docosanyl, lauroyl, stearoyl, palmitoyl, oleoyl, linoleoyl, other lipids, steroids, cholesterol, caffeine, vitamins, pigments, fluorescent substances, anticancer agent, toxin, enzymes, radioactive substance, biotin and the like. For such alterations, see, for example, U.S. Pat. Nos. 5,660,985 and 5,756,703.

However, the nucleic acid aptamer of the present invention may be an aptamer having any one of nucleotide sequences represented by SEQ ID NOs: 3 to 15.

The composition for detecting tebuconazole, mefenacet or inabenfide according to the present invention can be used to detect tebuconazole, mefenacet or inabenfide, which is most frequently detected among pesticides and herbicides that exceed maximum residue limits. Because residual pesticides may affect final consumers (humans) through various environmental pathways such as biological concentration even if they are present in food or environment in very small amounts, techniques for detecting and removing residual pesticides, which are widely used in agricultural and marine products, are required. Thus, for detection of tebuconazole, mefenacet or inabenfide, the nucleic acid aptamer of the present invention may be used in any form. For example, a DNA aptamer-tebuconazole, mefenacet or inabenfide complex prepared by immobilizing the nucleic acid aptamer to magnetic beads and bonding tebuconazole, mefenacet or inabenfide thereto can be separated using a magnet, and only tebuconazole, mefenacet or inabenfide can be selectively detected by separating tebuconazole, mefenacet or inabenfide from the complex.

In addition to the method described in an example of the present invention, which uses the nucleic aptamer-magnetic beads of the present invention, a method of detecting tebuconazole, mefenacet or inabenfide in a sample using a sensor connected to the nucleic acid aptamer of the present invention via a linker may be used.

In yet another aspect, the present invention provides a sensor for detecting tebuconazole, mefenacet or inabenfide, which contains the above-described aptamer that binds specifically to tebuconazole, mefenacet or inabenfide. The aptamer that binds specifically to tebuconazole, mefenacet or inabenfide may be immobilized on a substrate such as a chip to provide a sensor for detecting tebuconazole, mefenacet or inabenfide.

The sensor for detecting tebuconazole, mefenacet or inabenfide, which contains the aptamer that binds specifically to tebuconazole, mefenacet or inabenfide, may be provided in the form of a kit.

The kit for detecting tebuconazole, mefenacet or inabenfide may take the form of bottles, tubs, sachets, envelops, tubes, ampoules, and the like, which may be formed in part or in whole from plastic, glass, paper, foil, wax, and the like. The container may be equipped with a fully or partially detachable lid that may initially be part of the container or may be affixed to the container by mechanical, adhesive, or other means. The container may also be equipped with a stopper, allowing access to the contents by a syringe needle. The kit may comprise an exterior package which may include instructions regarding the use of the components.

It will be obvious to those skilled in the art that, because the nucleic acid aptamer of the present invention, which binds specifically to tebuconazole, mefenacet or inabenfide, also specifically detects only tebuconazole, mefenacet or inabenfide, a composition for separating tebuconazole, mefenacet or inabenfide may be provided which contains the nucleic acid aptamer.

In a further aspect, the present invention is directed to a method of removing or separating tebuconazole, mefenacet or inabenfide using the aptamer that binds specifically to tebuconazole, mefenacet or inabenfide.

In an example of the method of removing tebuconazole, mefenacet or inabenfide using the using the composition of the present invention, only tebuconazole, mefenacet or inabenfide may be selectively removed or separated by filling a column with magnetic beads having the nucleic acid aptamer immobilized thereon and then passing a sample containing tebuconazole, mefenacet or inabenfide through the column.

### EXAMPLES

Hereinafter, the present invention will be described in further detail with reference to examples. It will be obvious to a person having ordinary skill in the art that these examples are illustrative purposes only and are not to be construed to limit the scope of the present invention. Thus, the substantial scope of the present invention will be defined by the appended claims and equivalents thereof.

### Example 1

### Synthesis of DNA Pool Having Random Nucleotide Sequence

A 56-mer DNA pool having PCR primer regions at both ends and random nucleotides in its center was synthesized in the following manner. The DNA pool used in the present invention was chemically synthesized by Genotech Inc. (Korea).

CGTACGGAATTCGCTAGC-random region-GGATCCGAGCTCCACGTG
SEQ ID NO: 1:
CGTACGGAATTCGCTAGC
SEQ ID NO: 2:
GGATCCGAGCTCCACGTG

### Example 2

### Selection of DNA Aptamer Binding to All Tebuconazole, Mefenacet and Inabenfide

A random DNA pool and a counter target (pencycuron and Butachlor) were added to a buffer solution (20 mM Tris-Cl buffer, pH 7.6 contained 100 mM NaCl, 2 mM MgCl₂, 5 m MKCl, 1 mM CaCl₂, 0.02% Tween20, 10% MeOH), mixed with each other, and allowed to react at room temperature for 30 minutes. Then, to isolate a DNA unbound to the counter target, the mixture was reacted with a graphene oxide solution at room temperature for 30 minutes. At this time, a single-stranded DNA unbound to the counter target was strongly adsorbed onto the surface of the graphene through π-stacking. A DNA bound to the counter target was removed by centrifugation. To isolate a DNA that binds specifically to tebuconazole, mefenacet and inabenfide, tebuconazole, mefenacet and inabenfide were added to the tube containing graphene, and then allowed to react at room temperature for 30 minutes to thereby induce a conformational change, thereby separating a target-specific aptamer from the graphene. Next, the target-specific DNA was recovered by an ethanol precipitation method. The amount of the DNA binding specifically to tebuconazole, mefenacet and inabenfide, obtained as described above, was measured.

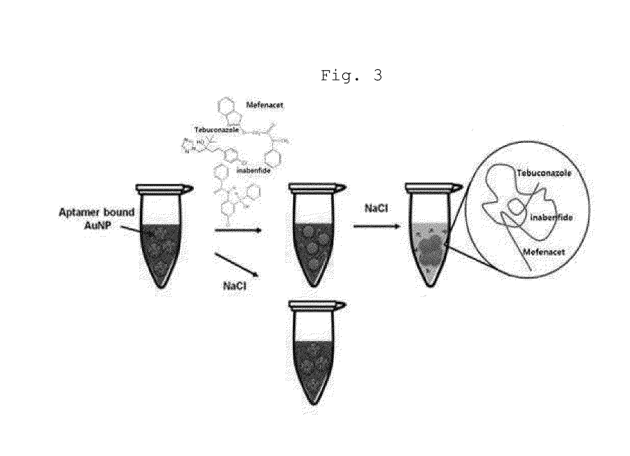

As shown in FIG. 2, the amounts of DNAs binding to tebuconazole, mefenacet and inabenfide, obtained in 1 to 3 selection rounds, increased.

### Example 3

### Selection of DNA Aptamer Capable of Binding Specifically to Tebuconazole

The DNA pool obtained in Example 2 and a counter target (pencycuron, Butachlor, mefenacet, and inabenfide) were added to a buffer solution (20 mM Tris-Cl buffer, pH 7.6 contained 100 mM NaCl, 2 mM MgCl₂, 5 mM KCl, 1 mM CaCl₂, 0.02% Tween20, 10% MeOH), mixed with each other, and allowed to react at room temperature for 30 minutes. Then, to isolate a DNA unbound to the counter target, the mixture was reacted with a graphene oxide solution at room temperature for 30 minutes. At this time, a single-stranded DNA unbound to the counter target was strongly adsorbed onto the surface of the graphene through π-stacking. A DNA bound to the counter target was removed by centrifugation. To isolate a DNA that binds specifically to tebuconazole, tebuconazole was added to the tube containing graphene, and then allowed to react at room temperature for 30 minutes to thereby induce a conformational change, thereby separating a target-specific aptamer from the graphene. Next, the target-specific DNA was recovered by an ethanol precipitation method. The amount of the DNA binding specifically to tebuconazole, obtained as described above, was measured. As shown in FIG. 2, the amount of the DNA binding to tebuconazole, obtained in each selection round, increased.

### Example 4

### Selection of DNA Aptamer Capable of Binding Specifically to Mefenacet

The DNA pool obtained in Example 2 and a counter target (pencycuron, Butachlor, tebuconazole, and inabenfide) were added to a buffer solution (20 mM Tris-Cl buffer, pH 7.6 contained 100 mM NaCl, 2 mM MgCl₂, 5 mM KCl, 1 mM CaCl₂, 0.02% Tween20, 10% MeOH), mixed with each other, and allowed to react at room temperature for 30 minutes. Then, to isolate a DNA unbound to the counter target, the mixture was reacted with a graphene oxide solution at room temperature for 30 minutes. At this time, a single-stranded DNA unbound to the counter target was strongly adsorbed onto the surface of the graphene through 90 -stacking. A DNA bound to the counter target was removed by centrifugation. To isolate a DNA that binds specifically to mefenacet, mefenacet was added to the tube containing graphene, and then allowed to react at room temperature for 30 minutes to thereby induce a conformational change, thereby separating a target-specific aptamer from the graphene. Next, the target-specific DNA was recovered by an ethanol precipitation method. The amount of the DNA binding specifically to mefenacet, obtained as described above, was measured. As shown in FIG. 2, the amount of the DNA binding to mefenacet, obtained in each selection round, increased.

### Example 5

### Selection of DNA Aptamer Capable of Binding Specifically to Inabenfide

The DNA pool obtained in Example 2 and a counter target (pencycuron, Butachlor, tebuconazole, and mefenacet) were added to a buffer solution (20 mM Tris-Cl buffer, pH 7.6 contained 100 mM NaCl, 2 mM MgCl₂, 5 mM KCl, 1 mM CaCl₂, 0.02% Tween20, 10% MeOH), mixed with each other, and allowed to react at room temperature for 30 minutes. Then, to isolate a DNA unbound to the counter target, the mixture was reacted with a graphene oxide solution at room temperature for 30 minutes. At this time, a single-stranded DNA unbound to the counter target was strongly adsorbed onto the surface of the graphene through π-stacking. A DNA bound to the counter target was removed by centrifugation. To isolate a DNA that binds specifically to inabenfide, inabenfide was added to the tube containing graphene, and then allowed to react at room temperature for 30 minutes to thereby induce a conformational change, thereby separating a target-specific aptamer from the graphene. Next, the target-specific DNA was recovered by an ethanol precipitation method. The amount of the DNA binding specifically to inabenfide, obtained as described above, was measured. As shown in FIG. 2, the amount of the DNA binding to mefenacet, obtained in each selection round, increased.

### Example 6

### Preparation of DNA Aptamer Pool Capable of Binding to Tebuconazole, Mefenacet and Inabenfide

In order to increase the amount of DNA binding specifically to tebuconazole, mefenacet and inabenfide, PCR was performed using already known primer regions.

Because the PCR product was a double-stranded DNA, one of the primers was labeled with fluorescein in order to separate the double-stranded DNA into single strands.

forward (APTFf)
SEQ ID NO: 16
5′-fluorescein-CGTACGGAATTCGCTAGC:
reverse (APTR)
SEQ ID NO: 17
5′-CACGTGGAGCTCGGATCC-3′:

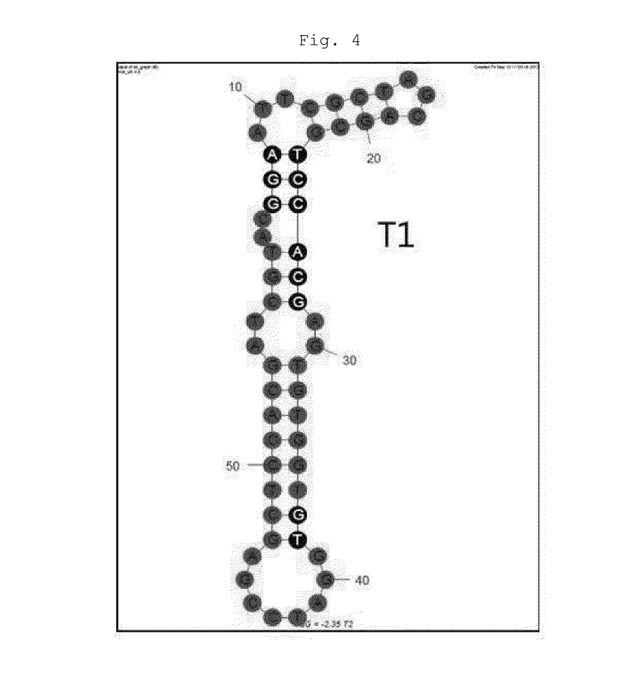

The PCR product was purified using a purification kit, and then subjected to polyacrylamide gel electrophoresis in order to separate the double-stranded DNA into single strands. 10% polyacrylamide gel contained 6M urea and 20% formamide, and thus two bands were produced after electrophoresis. In other words, in the electrophoresis, the double-stranded DNA was denatured, and thus the fluorescein-labeled DNA strand was located at the top, and the unlabeled DNA strand was located at the bottom. The fluorescein-labeled DNA band was cut and subjected to gel extraction, and then the DNA was recovered by an ethanol precipitation method. The obtained DNA pool was mixed with a solution of magnetic beads having immobilized thereon tebuconazole, mefenacet and inabenfide, and was allowed to react with tebuconazole, mefenacet and inabenfide. This process is schematically shown in FIG. 3. A total of six selection rounds, each consisting of a series of processes (FluMag-SELEX process), were performed, thereby obtaining a DNA pool, 60% or more of which binds to tebuconazole, mefenacet and inabenfide. After the third-round selection, counter selection was performed using AMPA, tebuconazole, mefenacet and inabenfide, thereby blocking a DNA that binds non-specifically to AMPA, and obtaining a DNA pool that binds specifically to tebuconazole, mefenacet and inabenfide with high affinity. The resulting DNA pool was cloned using a Quiagen cloning kit, and DNAs were extracted from the obtained colonies and sequenced. As a result, 13 different nucleic acid structures were obtained which bind specifically to tebuconazole, mefenacet or inabenfide.

### Example 7

### Analysis of Nucleotide Sequences and Specificities of 13 Different DNA Aptamers

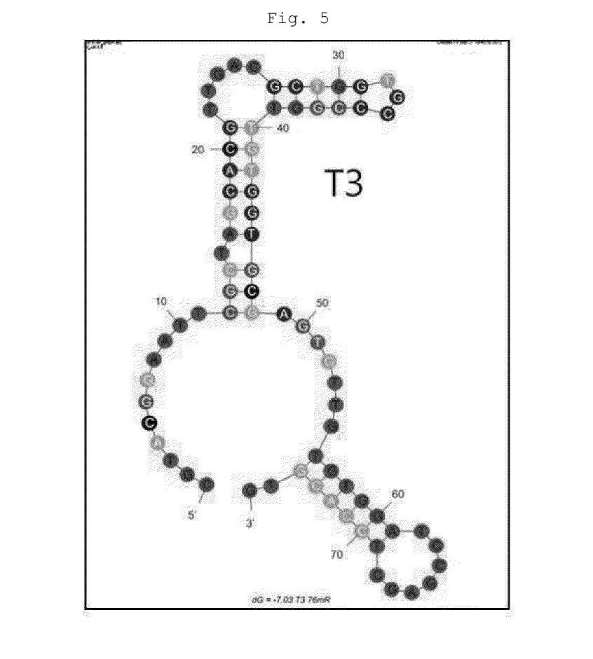

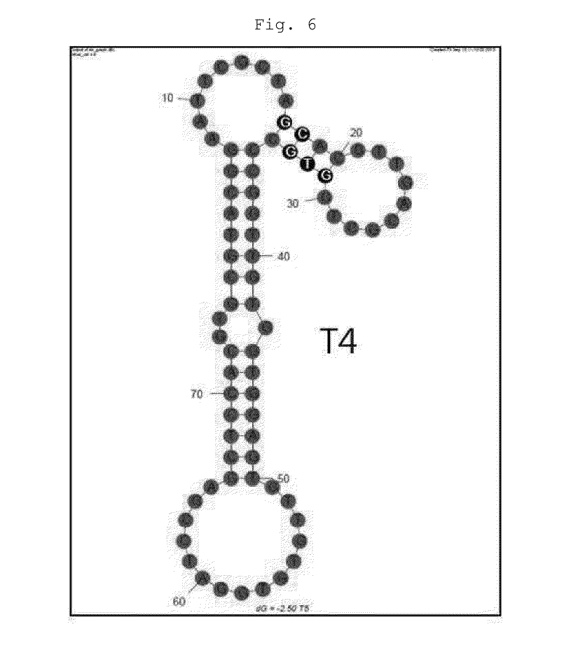

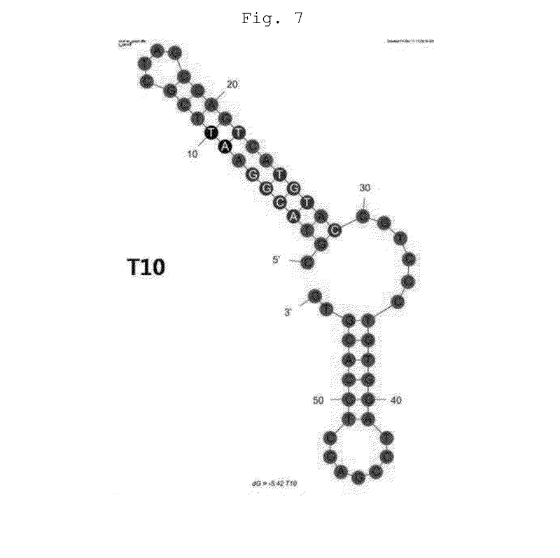

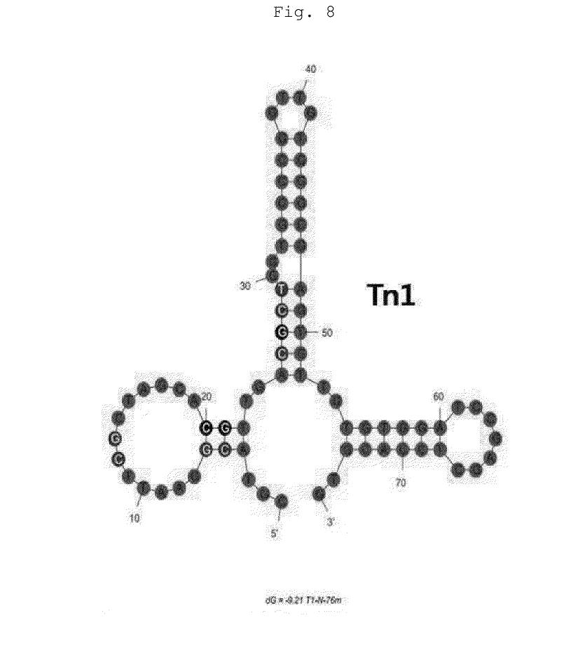

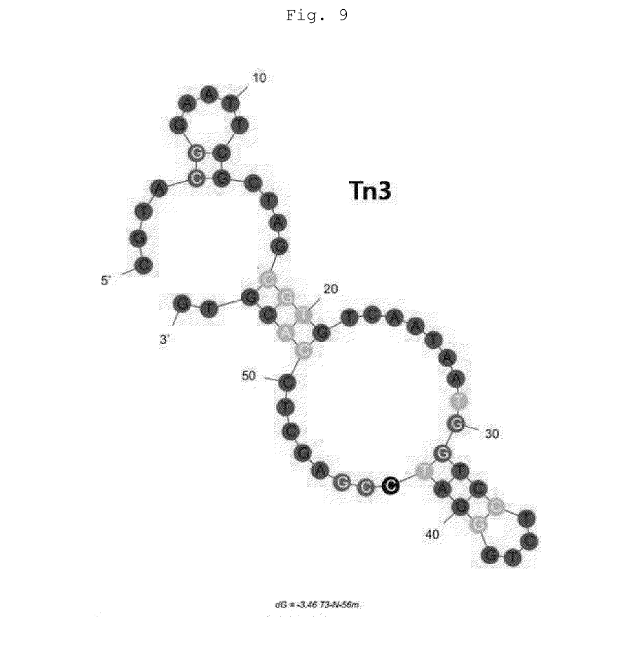

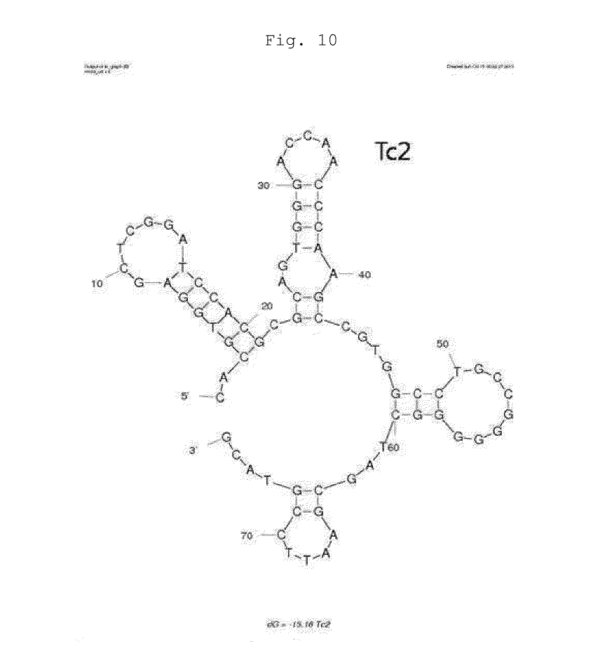

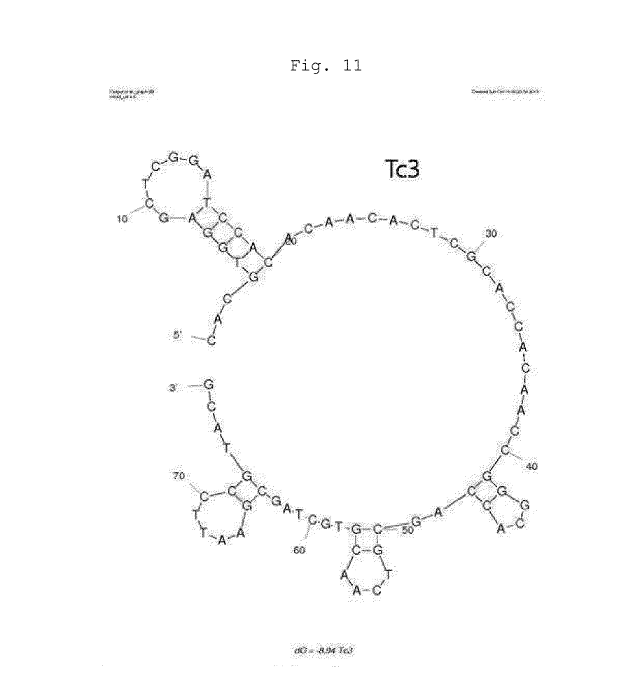

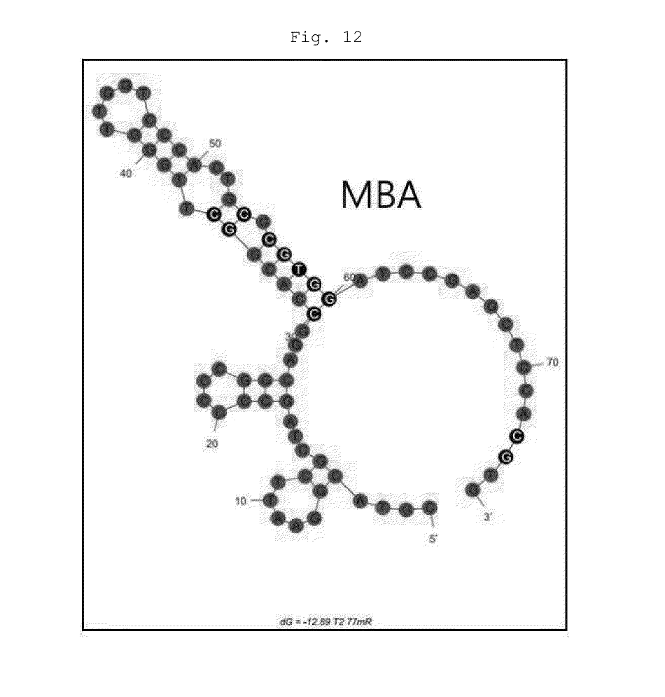

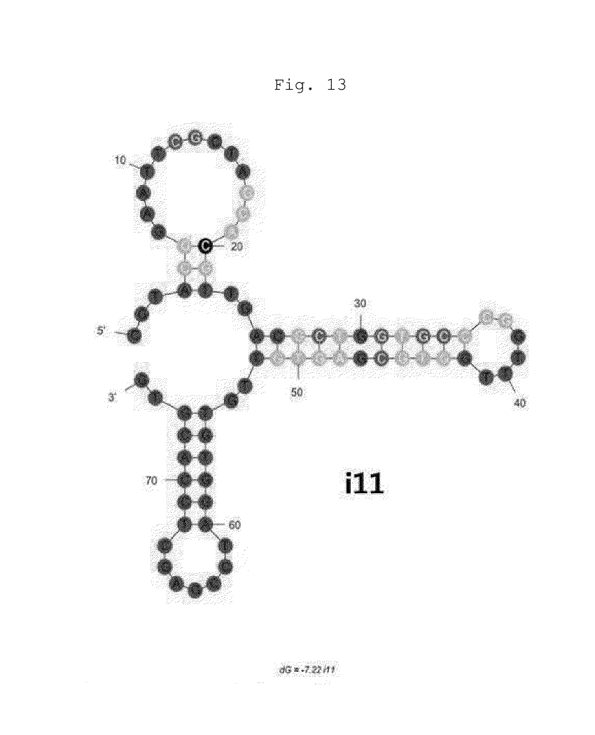

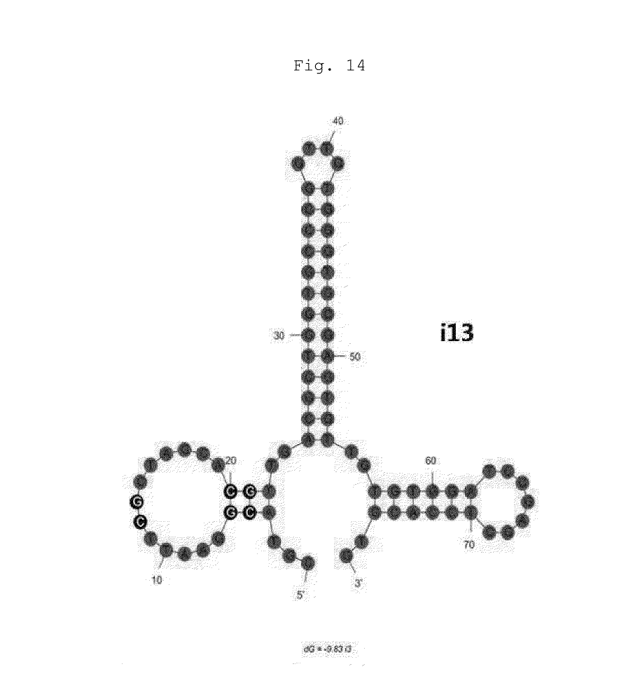

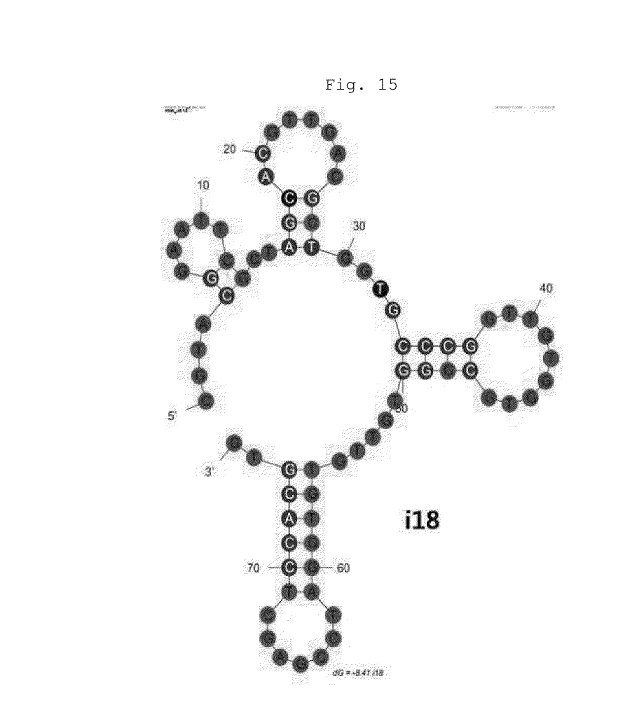

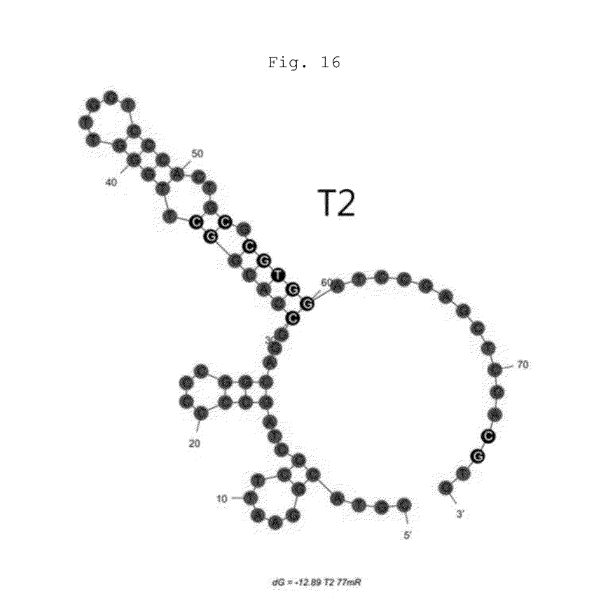

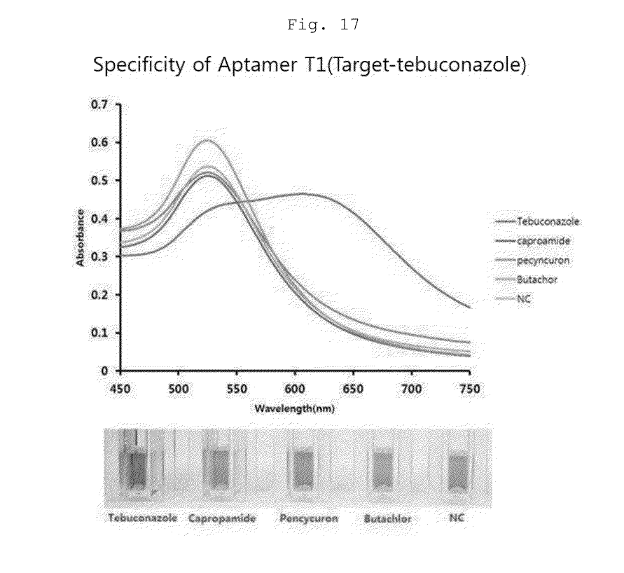

Table 1 below shows the results of analyzing the nucleotide sequences of 13 different DNAs that bind specifically to tebuconazole, mefenacet or inabenfide with high affinity. In addition, FIGS. 4 to 16 show the results of predicting secondary structures of the 13 aptamers specific for tebuconazole, mefenacet or inabenfide using m-fold program.

TABLE 1

| SEQ ID | | |
|---|---|---|
| NOS: | Aptamer | Sequences(5′-3) |
| 3 | T1 | CGTACGGAATTDGCTAGCAGCGTDCADGAGTGTG |
| | | GTGTGGATDCGAGCTDCADGAT |
| 4 | T3 | CGTACGGAATTDGCTAGCADGTTGADGCTGGTGD |
| | | CDGGTTGTGGTGCGAGTGTTGTGTGGATCDGAGC |
| | | TDCACGTG |
| 5 | T4 | CGTACGGAATTDGCTAGCADGTTGADGCTGGTGD |
| | | CDGGTTGTGGTGGAGTGTTGTGTGGATCCGAGCT |
| | | DCADGTG |
| 6 | T10 | CGTACGGAATTDGCTAGCGAGTCATGTADCGTDD |
| | | CTGTGGATDCGAGCTDCADGTG |
| 7 | Tn1 | CGTACGGAATTDGCTAGCADGTTGADGCTGGTGD |
| | | CDGGTTGTGGGDGAGTGTTGTGTGGATCCGAGCT |
| | | DCADGTG |
| 8 | Tn3 | CGTACGGAATTDGCTAGCGTGTCAATAATGGTDC |
| | | TCTGGGATDCGAGCTDCADGTG |
| 9 | Tc2 | CACGTGGAGCTDGGATDCADGDGCAGTGGGACCA |
| | | ADCCAAGCDGTGGDCTGDDGGGGGGCTAGDGAAT |
| | | TDCGTACG |
| 10 | Tc3 | CACGTGGAGCTDGGATDCACACAACACTDGCADC |
| | | ACAADCGGGCADCAGDGTCAADGTGCTAGDGAAT |
| | | TDCGTACG |
| 11 | i11 | CGTACGGAATTDGCTAGCADGTTGADGCTGGTGD |
| | | CDGGTTTGGTGDGAGTGTTGTGTGGATCCGAGCT |
| | | DCADGTG |
| 12 | i13 | CGTACGGAATTDGCTAGCADGTTGADGCTGGTGD |
| | | CDGGTTGTGGGTGDGAGTGTTGTGTGGATDDGAG |
| | | CTCCADGTG |
| 13 | i18 | CGTACGGAATTDGCTAGCADGTTGADGCTGGTGD |
| | | CDGGTTGTGGTGCGGGTGTTGTGTGGATCDGAGC |
| | | TDCACGTG |
| 14 | MBA | CGTACGGAATTDGCTAGCDDCDCGGCAGGCCADG |
| | | GCTTGGGTTGGTCDCACTGDGDGTGGATCDGAGC |
| | | TDCACGTG |
| 15 | T2 | CGTACGGAATTCGCTAGCCCCCCGGCAGGCCACG |
| | | GCTTGGGTTGGTCCCTCTGCGCGTGGATCCGAGC |
| | | TCCACGTG |

Among the 13 DNA aptamers, an aptamer having high specificity for each of tebuconazole, mefenacet and inabenfide as a target was selected in the following manner.

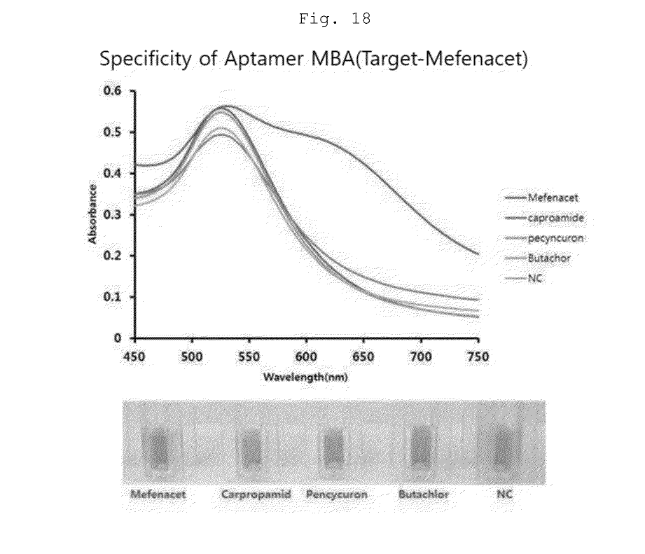

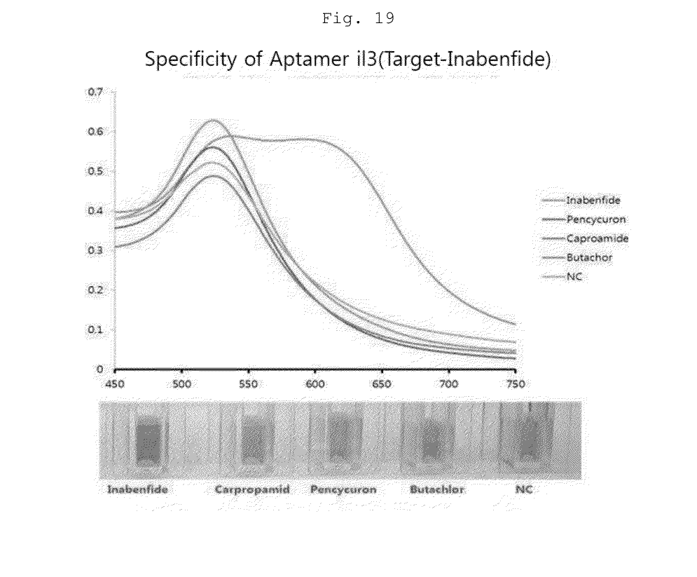

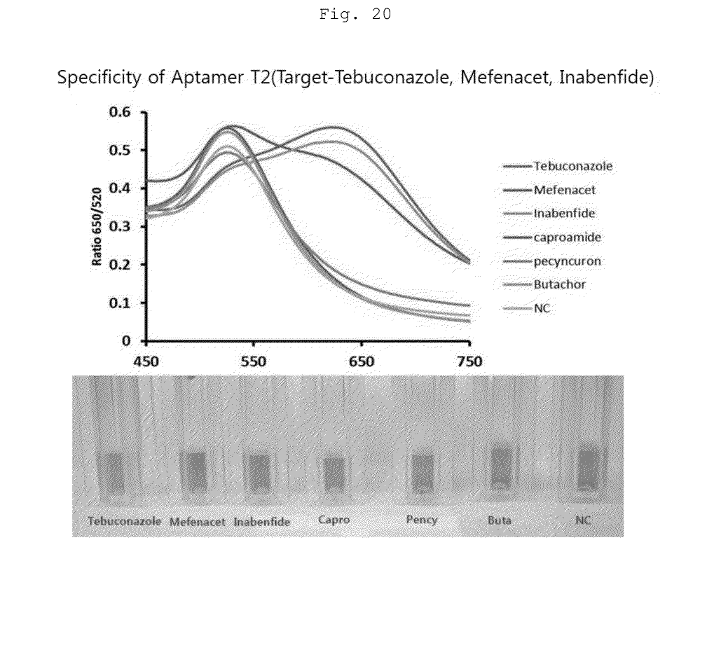

2 nM of gold nanoparticles and 200 nM of the aptamer binding to each of tebuconazole, mefenacet and inabenfide were added to triple-distilled water and subjected to an adsorption reaction at room temperature for 30 minutes. Next, 250 uM of each of tebuconazole (Formula 1), mefenacet (Formula 2), and inabenfide (Formula 3), carpropamid (Formula 4), pencycuron (Formula 5) and Butachlor (Formula 6) was added to the reaction product and allowed to react for 30 minutes. Then, 0.6M NaCl was added to each of the reaction solutions to induce salt-induced gold aggregation, and then the UV absorbance of the gold nanoparticle solutions was measured. Based on the results of the measurement, aptamer T1 having the highest binding affinity for the target was selected from the aptamers. FIGS. 17 to 20 show a UV absorbance graph and a photograph of the samples after the reaction.

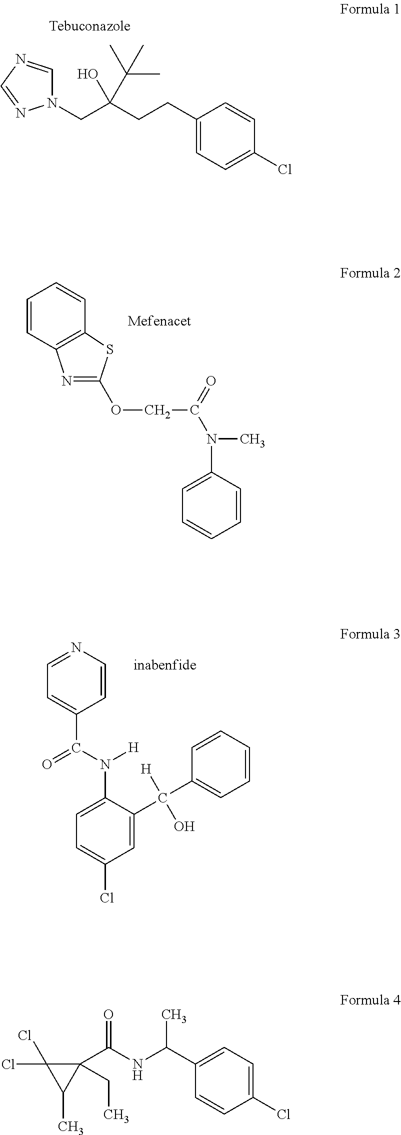 [SMILES: CC(C)(C)C(O)(CCc1ccc(Cl)cc1)Cn1cncn1 ; CN(C(=O)COc1nc2ccccc2s1)c1ccccc1 ; O=C(Nc1ccc(Cl)cc1C(O)c1ccccc1)c1ccncc1 ; CCC1(C(=O)NC(C)c2ccc(Cl)cc2)C(C)C1(Cl)Cl]

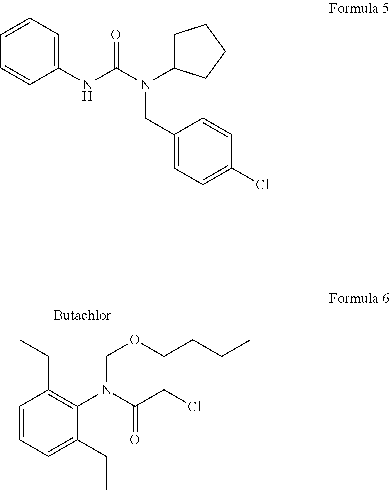 [SMILES: O=C(Nc1ccccc1)N(Cc1ccc(Cl)cc1)C1CCCC1 ; CCCCOCN(C(=O)CCl)c1c(CC)cccc1CC]

### Example 8

### Analysis of Binding Affinity of DNA Aptamer for Tebuconazole

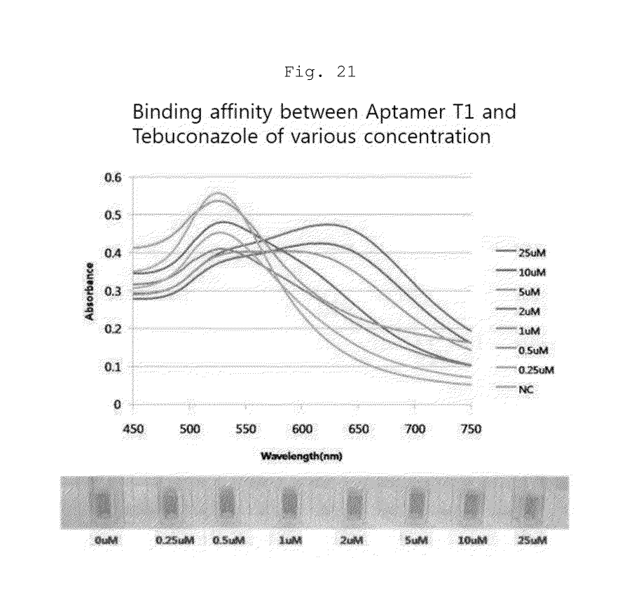

Among the 13 different aptamers that bind specifically to tebuconazole, aptamer T1 having the highest specificity was selected, and the binding affinities of aptamer T1 for various concentrations of tebuconazole were analyzed. Specifically, 2 nM of gold nanoparticles and 200 nM of the aptamer binding to each of tebuconazole, mefenacet and inabenfide were added to triple-distilled water and allowed to react at room temperature for 30 minutes, after which 0 to 25 uM of tebuconazole was added thereto and allowed to react for 30 minutes. 0.6M NaCl was added to the reaction solution to induce salt-induced gold aggregation, and the UV absorbance of the gold nanoparticles was measured. FIG. 21 shows a UV absorbance graph and a photograph of the samples after the reaction. As can be seen in FIG. 21, tebuconazole can be detected using the tebuconazole-specific aptamer and gold nanoparticles.

### INDUSTRIAL APPLICABILITY

As described above, the nucleic acid aptamer of the present invention, which binds specifically to tebuconazole, mefenacet or inabenfide, enables the detection and separation of a very small amount of tebuconazole, mefenacet or inabenfide, which is present in soil, water or food. Accordingly, the nucleic acid aptamer of the present invention enables the detection of vary small amounts of residual pesticides present in food or environment, and thus can be used to protect humans from biological concentration or the like. In addition, because the nucleic acid aptamer of the present invention can be produced at low costs, a method and kit for detecting tebuconazole, mefenacet or inabenfide, and a kit for separating tebuconazole, mefenacet or inabenfide, which use the nucleic acid aptamer of the present invention, is cost-effective and highly useful.

From the foregoing, it will be understood by those skilled in the art to which the present invention pertains that the present invention can be carried out in other concrete embodiments without changing the technical spirit or essential feature thereof In this regard, it should be understood that the aforementioned examples are of illustrative in all aspects but not is limited. The scope of the present invention should be construed to include the meaning and scope of the appended claims, and all the alterations and modified forms which are derived from the equivalent concept thereof, rather than the detailed description.

1. A nucleic acid aptamer capable of binding specifically to tebuconazole, mefenacet or inabenfide, which has a nucleotide sequence selected from the group consisting of SEQ ID NOs: 3 to 15,
wherein if the nucleic acid is RNA, “T” in the nucleic acid sequence is to be read as “U”.

2. A method for detecting tebuconazole, mefenacet or inabenfide, the method comprises contacting the nucleic acid aptamer of claim 1 with a sample containing tebuconazole, mefenacet or inabenfide.

3. The method of claim 2, wherein the sample is collected from one or more of water, soil, waste, food, animal intestines, and animal and plant tissues.

4. The method of claim 2, wherein the detection of tebuconazole, mefenacet or inabenfide is performed by a gold nanoparticle-based colorimetric assay.

5. A composition for detecting tebuconazole, mefenacet or inabenfide, which contains the aptamer of claim 1.

6. A sensor for detecting tebuconazole, mefenacet or inabenfide, which contains the aptamer of claim 1.

7. A kit for detecting tebuconazole, mefenacet or inabenfide, which contains the aptamer of claim 1.

8. A method of separating tebuconazole, mefenacet or inabenfide from a sample using the aptamer of claim 1.

9. The method of claim 8, wherein the sample is passed through a column filled with beads having the nucleic acid aptamer immobilized thereon.

10. The method of claim 8, wherein the sample is collected from one or more of water, soil, waste, food, animal intestines, and animal and plant tissues.

11. A kit for separating tebuconazole, mefenacet or inabenfide, which contains the aptamer of claim 1.
